(19) United States
(12) Patent Application Publication
(10) Pub. No.: US 2005/0065212 A1
(43) Pub. Date: Mar. 24, 2005

(54) Use of at least one 10-hydroxy-2-decenoic acid derivative in compositions for treating the cuteous signs of aging

(76) Inventors: Nathalie Pineau, Corneilles-En-Parisis (FR); Dominique Fagot, Paris (FR); Emile Benechie, Gif-Sur-Yvette (FR); Martin Li, Le Plessis Robinson (FR); Francoise Picot, Chevreuse (FR); Pierre Potier, Paris (FR)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 10/480,502

(22) PCT Filed: Jun. 14, 2002
(86) PCT No.: PCT/FR02/02066

(30) Foreign Application Priority Data
Jun. 14, 2001 (FR) 0107803

(51) Int. Cl. A61K 31/225; A61K 31/22

(52) U.S. Cl. 514/547; 514/549

(57) ABSTRACT

The present invention relates to a method of inhibiting degradation of skin or mucous membranes by inhibiting action of collagenases comprising applying to the skin or mucous membranes a composition comprising at least one of 10-hydroxy-2-decenoic acid derivative. The invention also relates to a process for treating signs of aging.

### BACKGROUND OF THE INVENTION

Human skin consists of two compartments, namely a surface compartment, the epidermis, and a deep compartment, the dermis.

Natural human epidermis is composed mainly of three types of cells: keratinocytes, which form the great majority, melanocytes and Langerhans cells. Each of these cell types contributes, by virtue of its intrinsic functions, toward the essential role played in the body by the skin.

The dermis gives the epidermis a solid support. It is also the epidermis' nourishing factor. It consists mainly of fibroblasts and of an extracellular matrix which is itself composed mainly of collagen, elastin and a substance known as ground substance, these components being synthesized by the fibroblasts. Leukocytes, mastocytes and tissue macrophages are also found therein. It also contains blood vessels and nerve fibers. In normal skin, that is to say non-pathological and unscarred skin, the fibroblasts are in the quiescent state, i.e. non-proliferative, metabolically relatively inactive, and immobile.

These collagen fibers give the dermis its firmness. Collagen fibers consist of fibrils sealed together, thus forming more than 10 different types of structures. The firmness of the dermis is mainly due to the entanglement of the collagen fibers packed together in all directions. The collagen fibers contribute toward the elasticity and tonicity of the skin and/or mucous membranes.

The collagen fibers are under constant renewal, but this renewal decreases with age, leading to thinning of the dermis. This thinning of the dermis is also due to pathological causes such as, for example, the hypersecretion of corticoid hormones, certain pathologies or vitamin deficiencies (which is the case for vitamin C in scurvy). It is also accepted that extrinsic factors such as ultraviolet rays, tobacco or certain treatments (glucocorticoids, vitamin D and derivatives, for example) also have an effect on the skin and its collagen content.

However, various factors lead to the degradation of collagen, with all the consequences which may be envisaged on the structure and/or firmness of the skin and/or mucous membranes.

Although very strong, collagen fibers are sensitive to certain enzymes known as collagenases. A degradation of collagen fibers leads to the development of flaccid and wrinkled skin, which people, preferring the appearance of smooth and taut skin, have always sought to combat.

Collagenases form part of a family of enzymes known as metalloproteases (MMPs) which are themselves members of a family of proteolytic enzymes (endoproteases or endopeptidases) which contain a zinc atom coordinated to 3 cysteine residues and one methionine residue in their active site and which degrade the macromolecular components of the extracellular matrix and of the basal membranes at neutral pH (collagen, elastin, etc.). These enzymes, which are very widespread in the living world, are present, but poorly expressed, in normal physiological situations such as the growth of organs and the renewal of tissues.

However, their overexpression in man and their activation are linked to many processes, sometimes pathological processes, which involve the destruction and remodeling of the matrix. This results in either an uncontrolled resorption of extracellular matrix or, conversely, the installation of a state of fibrosis.

The metalloprotease family consists of several well-defined groups based on their resemblances in terms of structure and substrate specificity (see Woessner J. F., Faseb Journal, vol. 5, 1991, 2145). Among these groups, mention may be made of the collagenases for degrading fibrillar collagens (MMP-1 or interstitial collagenase, MMP-8 or neutrophil collagehase and MMP-13 or collagenase 3), gelatinases which degrade type IV collagen or any form of denatured collagen (MMP-2 or gelatinase A (72 kDa), MMP-9 or gelatinase B (92 kDa)), stromelysins (MMP-3) whose broad spectrum of activity addresses the proteins of the extracellular matrix such as glycoproteins (fibronectin, laminin), proteoglycans, etc., or alternatively membrane metalloproteases.

Prolonged exposure to ultraviolet rays, particularly to ultraviolet rays of A and/or B type, has the effect of stimulating the expression of collagenases, particularly of MMP-1. This is one of the components of photo-induced aging of the skin.

Moreover, at the menopause the main changes concerning the dermis are a decrease in collagen content and in the thickness of the dermis. In menopausal women, this results in thinning of the skin and/or mucous membranes. Women then experience a sensation of “dry skin” or of taut skin and there is an increase in surface wrinkles and fine lines. The skin has a rough aspect when touched. Finally, the skin is less supple.

The importance of collagen in the structure of tissues, particularly the skin and/or mucous membranes, and the importance of combating its degradation in order thus to combat aging, whether this is chronobiological or photo-induced aging, and the consequences thereof, the thinning of the dermis and/or the degradation of collagen fibers which result in the development of flaccid and wrinkled skin, may thus be appreciated on reading the account hereinabove.

### SUMMARY OF THE INVENTION

One of the aims of the present invention is thus to be able to provide a novel product which has an inhibitory effect on collagenases and, if possible, no appreciable side effects.

Surprisingly and unexpectedly, the Applicant has now discovered that certain particular 10-hydroxy-2-decenoic acid derivatives have inhibitory activity on the action of collagenases, via inhibition of the production and/or synthesis of collagenases. The expression “inhibition of the production” should be understood as meaning inhibition of the signaling pathways leading to the expression of collagenase messenger RNA and/or inhibition of the translation of the collagenase messenger RNA into proteins and/or inhibition of the secretion of the collagenases outside the collagenase-producing cell.

In the publications of the prior art, 10-hydroxy-2-decenoic acid derivatives are known for their property of activating the immune system.

To the Applicant's knowledge, it has never been described in the prior art that 10-hydroxy-2-decenoic acid derivatives have inhibitory activity on the action of collagenases.

Consequently, the Applicant is proposing the use of certain particular 10-hydroxy-2-decenoic acid derivatives to inhibit the activity of collagenases and thus to combat collagen degradation, thus making it possible to treat the signs of aging of the skin associated with collagen degradation.

### DETAILED DESCRIPTION

The expression “signs of aging of the skin associated with collagen degradation” means any change in the external appearance of the skin due to collagen degradation, such as, for example, wrinkles and fine lines, withered skin, thinned skin, and skin lacking elasticity and/or tonus, and also any internal change in the skin which is not systematically reflected by a changed external appearance.

A first subject of the invention is thus the use, in a composition, of an effective amount of 10-hydroxy-2-decenoic acid derivatives having the formula described below, the derivative or the composition being intended to inhibit the expression of proteases of the extracellular matrix, particularly metalloproteases and even more particularly the type-1 matrix metalloprotease (MMP-1). The invention thus relates to compounds derived from 10-hydroxy-2-decenoic acid, having the following formulae:
2-dimethylaminoethyl 10-hydroxy-2-decenoate of formula: 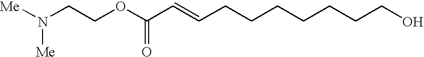
2,3-dihydroxypropyl 10-hydroxy-2-decenoate of formula: 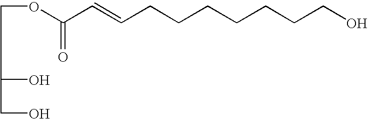
2-hydroxypropyl 1,3-bis(10-hydroxy-2-decenoate) of formula: 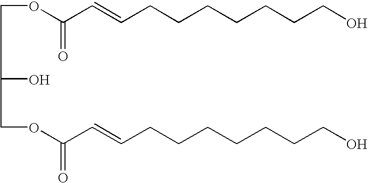
propyl 1,2,3-tris(10-hydroxy-2-decenoate) of formula: 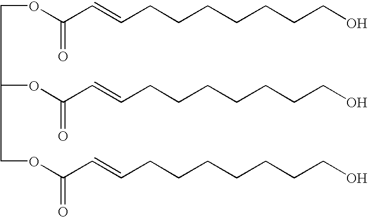
3-[(2-methoxy)ethoxymethoxy]propyl 1,2-bis(10-hydroxy-2-decenoate) of formula:

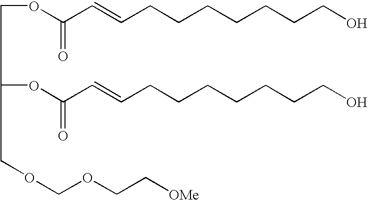

In the text hereinbelow, the term “10-hydroxy-2-decenoic acid derivatives” is understood as denoting strictly the compounds described above, of natural or synthetic origin, in purified form or any preparation containing them.

The term “natural origin” means a compound extracted from natural material in which it is present. The term “synthetic origin” means a compound prepared by chemical synthesis or by biotechnology.

Needless to say, the 10-hydroxy-2-decenoic acid derivatives may be used according to the invention alone or as a mixture.

Similarly, the 10-hydroxy-2-decenoic acid derivatives may be used in their cis and/or trans form.

Finally, the use according to the invention may be envisaged curatively and/or preventively.

According to the invention, the term “inhibition” means totally or partially reducing the level of production, expression and/or translation and/or secretion of the proteases of the extracellular matrix.

Preferably, according to the invention, the use of at least one 10-hydroxy-2-decenoic acid derivative is intended to combat collagen degradation and very preferentially to treat the signs of aging of the skin associated with collagen degradation, particularly during the menopause.

Among the skin complaints associated with collagen degradation, the invention is particularly directed toward treating wrinkles and fine lines, withered skin, flaccid skin, thinned skin or skin lacking elasticity and/or tonus.

The 10-hydroxy-2-decenoic acid derivative may by itself constitute the active principle of the compositions of the invention.

The amount of 10-hydroxy-2-decenoic acid derivatives that may be used according to the invention obviously depends on the desired effect and should be in an amount that is effective to inhibit the expression and/or translation and/or secretion of the proteases of the extracellular matrix.

By way of example, the amount of 10-hydroxy-2-decenoic acid derivatives that may be used according to the invention may range, for example, from 0.001% to 10% and preferably from 0.01% to 1% of the total weight of the composition.

The composition of the invention may be in any imaginable presentation form suitable for topical application to the skin and/or mucous membranes and/or the hair.

Preferably, the composition of the invention is intended for topical administration.

The composition of the invention may be a cosmetic or dermatological composition. Preferably, according to the invention, the composition is a cosmetic composition. The composition is a cosmetic composition since it is intended to improve the general appearance of the skin of the individual using it.

Very preferably, the composition of the invention is a cosmetic composition for topical administration.

For an administration by topical application to the skin, hair and/or mucous membranes, the composition according to the invention obviously comprises a cosmetically acceptable medium, that is to say a medium which is compatible with the skin, mucous membranes, the nails or the hair, and may be in any pharmaceutical form normally used for topical application, preferably in the form of an aqueous, aqueous-alcoholic or oily solution, an oil-in-water or water-in-oil or multiple emulsion, an aqueous or oily gel, an anhydrous liquid, pasty or solid product, or a dispersion of oil in an aqueous phase with the aid of spherules, wherein these spherules are preferably polymer nanoparticles such as nanospheres and nanocapsules or more preferably lipid vesicles of ionic and/or nonionic type.

This composition may be more or less fluid and may have the appearance of a white or colored cream, an ointment, a milk, a lotion, a serum, a paste or a mousse. It may optionally be applied to the skin in the form of an aerosol. It may also be in solid form, and for example in the form of a stick. It may be used as a care product, as a cleansing product, as a makeup product or as a simple deodorant product.

In a known manner, the composition of the invention may contain adjuvants that are common in cosmetics and dermatology, such as hydrophilic or lipophilic gelling agents, hydrophilic or lipophilic active agents, preserving agents, antioxidants, solvents, fragrances, fillers, sreening agents, pigments, chelating agents, odor absorbers and dyestuffs. The amounts of these various adjuvants are those conventionally used in the fields under consideration, and, for example, from 0.01% to 20% relative to the total weight of the composition. Depending on their nature, these adjuvants may be introduced into the fatty phase, into the aqueous phase, into lipid vesicles and/or into nanoparticles.

When the composition of the invention is an emulsion, the proportion of the fatty phase may range from 5% to 80% by weight and preferably from 5% to 50% relative to the total weight of the composition. The oils, emulsifiers and co-emulsifiers used in the composition in emulsion form are chosen from those conventionally used in the field under consideration. The emulsifier and co-emulsifier are present in the composition in a proportion ranging from 0.3% to 30% by weight and preferably from 0.5% to 20% relative to the total weight of the composition.

As oils which may be used in the invention, mention may be made of mineral oils, oils of plant origin (apricot oil or sunflower oil), oils of animal origin, synthetic oils, silicone oils and fluoro oils (perfluoropolyethers). Fatty alcohols (cetyl alcohol), fatty acids and waxes (beeswax) may also be used as fatty substances.

As emulsifiers and coemulsifiers which may be used in the invention, mention may be made, for example, of fatty acid esters of polyethylene glycol, such as PEG-40 stearate and PEG-100 stearate, and fatty acid esters of polyols, such as glyceryl stearate and sorbitan tristearate.

Hydrophilic gelling agents which may be mentioned in particular are carboxyvinyl polymers (carbomer), acrylic copolymers such as acrylate/alkylacrylate copolymers, polyacrylamides, polysaccharides, natural gums and clays, and lipophilic gelling agents which may be mentioned are modified clays, for instance bentones, metal salts of fatty acids, hydrophobic silica and polyethylenes.

Another subject of the present invention is a cosmetic treatment process for treating signs of aging of the skin associated with collagen degradation, that is to say any change in the external appearance of the skin due to collagen degradation, such as, for example, wrinkles and fine lines, withered skin, flaccid skin, thinned skin, and skin lacking elasticity and/or tonus, and also any internal change in the skin which is not systematically reflected by a changed external appearance, for instance any internal degradation of the skin following exposure to ultraviolet radiation, characterized in that a cosmetic composition comprising at least one 10-hydroxy-2-decenoic acid derivative is applied to the skin, the hair and/or mucous membranes, or is ingested.

The cosmetic treatment process of the invention may be carried out especially by applying the cosmetic compositions as defined above, according to the usual technique for using these compositions. For example: application of creams, gels, sera, lotions, milks, shampoos or antisun compositions to the skin or to the hair, or alternatively application of toothpaste to the gums.

The examples and compositions below illustrate the invention without limiting it in any way. In the compositions, the proportions indicated are percentages by weight.

### EXAMPLES

### Example 1

### Effect of 2-dimethylaminoethyl 10-hydroxy-2-decenoate on the production of collagenases

The effect of 2-dimethylaminoethyl 10-hydroxy-2-decenoate on the production of interstitial collagenase was evaluated in a culture model of A2058 cells (derived from human melanomas: Templeton N. S. et al. 1990; Cancer Res., 50:5431-5431) and in a culture model of HT1080 cells derived from a human fibrosarcoma (American type Culture Collection: reference CCL 121).

The A2058 cells constitutively express and secrete type-1 metalloprotease. The HT1080 cells express little type-1 metalloprotease in the basal state, but this expression is increased in the presence of TPA.

The cells are incubated in an MEM medium without phenol red, containing amino acids at a concentration of 2 mM, sodium pyruvate at a concentration of 1 mM and calf serum containing 10% graphite. They are then cultured at a density of 20 000 cells per well in the 24 wells of a multiwell plate.

Twenty-four hours after culturing, the cells are placed in contact with 2-dimethylaminoethyl 10-hydroxy-2-decenoate at a concentration of 25 μg/ml. The production of the interstitial collagenase is evaluated 96 hours later in the culture medium. This is performed using an ELISA kit (Biotrack human MMP1; Amersham).

The results are expressed as a percentage of reduction of the production of interstitial collagenase relative to the control, i.e. relative to culturing performed under the same conditions in the absence of derivative.

| | | HT1080 | HT1080 with |
|---|---|---|---|
| | A2058 | without TPA | TPA |
| Compound at a | 70% | 55% | 50% |
| concentration of | | | |
| 25 μg/ml | | | |

The results show that the derivative at a dose of 25 μg/ml reduces the production of interstitial collagenase by the A 2058 cells and by the HT61080 cells.

### Example 2

Examples of formulations illustrating the invention and particularly the compositions according to the invention. These compositions were obtained according to the techniques conventionally used in the cosmetics industry.

| Composition 1: Facial care cream (oil-in-water | |
|---|---|
| emulsion) | |
| 2-Dimethylaminoethyl 10-hydroxy-2-decenoate | 1.00% |
| Glyceryl stearate | 2.00% |
| Polysorbate 60 (Tween 60 ® sold by the company | 1.00% |
| ICI) | |
| Stearic acid | 1.40% |
| Triethanolamine | 0.70% |
| Carbomer | 0.40% |
| Liquid fraction of karite butter | 12.00% |
| Perhydrosqualene | 12.00% |
| Antioxidant | 0.05% |
| Fragrance | 0.50% |
| Preserving agent | 0.30% |
| Water qs | 100.00% |
| Composition 2: Gel for the skin | |
| 2,3-Dihydroxypropyl 10-hydroxy-2-decenoate | 2.00% |
| All-trans-retinoic acid | 0.05% |
| Hydroxypropylcellulose (Klucel H ® sold by the | 1.00% |
| company Hercules) | |
| Antioxidant | 0.05% |
| Isopropanol | 40.00% |
| Preserving agent | 0.30% |
| Water qs | 100.00% |
| Composition 3: Facial care gel | |
| 2-Dimethylaminoethyl 10-hydroxy-2-decenoate | 2.50% |
| Hydroxypropylcellulose (Klucel H ® sold by the | 1.00% |
| company Hercules) | |
| Antioxidant | 0.05% |
| Isopropanol | 40.00% |
| Preserving agent | 0.30% |
| Water qs | 100.00% |
| Composition 4: Gel | |
| 2,3-Dihydroxypropyl 10-hydroxy-2-decenoate | 1.50% |
| Hydroxypropylcellulose (Klucel H ® sold by the | 1.00% |
| company Hercules) | |
| Antioxidant | 0.05% |
| Lidocaine hydrochloride | 2.00% |
| Isopropanol | 40.00% |
| Preserving agent | 0.30% |
| Water qs | 100.00% |
| Composition 5: Cream for treating solar | |
| erythema (oil-in-water emulsion) | |
| 2-Dimethylaminoethyl 10-hydroxy-2-decenoate | 1.00% |
| Glyceryl stearate | 2.00% |
| Polysorbate 60 (Tween 60 ® sold by the company | 1.00% |
| ICI) | |
| Stearic acid | 1.40% |
| Glycyrrhetinic acid | 2.00% |
| Triethanolamine | 0.70% |
| Carbomer | 0.40% |
| Liquid fraction of karite butter | 12.00% |
| Sunflower oil | 10.00% |
| Antioxidant | 0.05% |
| Fragrance | 0.50% |
| Preserving agent | 0.30% |
| Water qs | 100.00% |
| Composition 6: Facial anti-wrinkle care cream | |
| (oil-in-water emulsion) | |
| 2,3-Dihydroxypropyl 10-hydroxy-2-decenoate | 2.50% |
| Glyceryl stearate | 2.00% |
| Polysorbate 60 (Tween 60 ® sold by the company | 1.00% |
| ICI) | |
| Stearic acid | 1.40% |
| 5-n-Octanoylsalicylic acid | 0.50% |
| Triethanolamine | 0.70% |
| Carbomer | 0.40% |
| Liquid fraction of karite butter | 12.00% |
| Perhydrosqualene | 12.00% |
| Antioxidant | 0.05% |
| Fragrance | 0.50% |
| Preserving agent | 0.30% |
| Water qs | 100.00% |

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of inhibiting the expression, translation or secretion of proteases of the extracellular matrix in skin or mucous membranes comprising: applying to said skin or mucous membranes a composition comprising at least one 10-hydroxy-2-decenoic acid derivative chosen from:
2-dimethylaminoethyl 10-hydroxy-2-decenoate of formula: 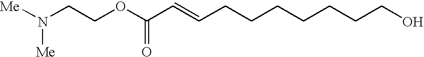
2,3-dihydroxypropyl 10-hydroxy-2-decenoate of formula: 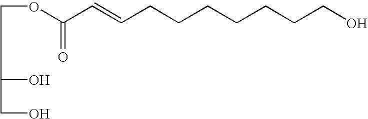
2-hydroxypropyl 1,3-bis(10-hydroxy-2-decenoate) of formula: 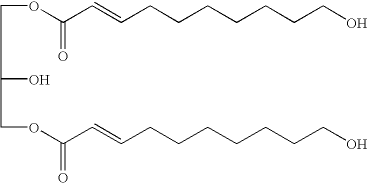
propyl 1,2,3-tris(10-hydroxy-2-decenoate) of formula: 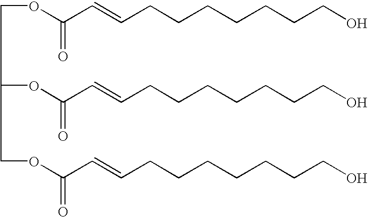
3-[(2-methoxy)ethoxymethoxy]propyl 1,2-bis (10-hydroxy-2-decenoate) of formula: 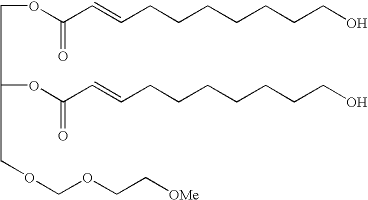
.

2. The method of claim 1, said composition inhibits the expression, translation or secretion of metalloproteases.

3. The method as in claim 1 or 2, wherein said composition inhibits the expression, translation or secretion of type-1 matrix metalloprotease.

4. The method as in claim 1 or 2, wherein said composition treats wrinkles and fine lines.

5. The method as in claim 1 or 2, wherein said composition treats withered skin.

6. The method as in claim 1 or 2, wherein said composition treats thinned skin.

7. The method as in claim 1 or 2, wherein said composition treats a lack of elasticity and/or tone of said skin.

8. The method as in claim 1 or 2, wherein said composition inhibits internal degradation of said skin caused by exposure to ultraviolet radiation.

9. The method of claim 8, wherein said composition inhibits collagen degradation.

10. A method of treating skin complaints associated with the menopause comprising applying to said skin a composition comprising at least one 10-hydroxy-2-decenoic acid derivative as described in claim 1.

11. The method as in claim 1 or 2, wherein said composition comprises between about 0.001% and about 10% of said at least one 10-hydroxy-2-decenoic acid derivative based on the total weight of the composition.

12. The method of claim 11, wherein said composition comprises between about 0.01% and about 1% of said at least one 10-hydroxy-2-decenoic acid derivative based on the total weight of the composition.

13. (cancelled)

14. A process for treating signs of aging of skin, hair or mucous membrane comprising: applying to said skin or ingesting a composition comprising at least one 10-hydroxy-2-decenoic acid derivative as described in claim 1 to said skin, hair or mucous membrane.

15. A process for treating the wrinkles and fine lines, withered skin, thinned skin, the lack of elasticity and/or tone of the skin or any internal change in the skin which is not systematically reflected by a changed external appearance, comprising: applying to said skin or ingesting a composition comprising at least one 10-hydroxy-2-decenoic acid derivative as described in claim 1.
